(19) United States
(12) Patent Application Publication
(10) Pub. No.: US 2022/0175303 A1
(43) Pub. Date: Jun. 9, 2022

(54) BRAIN-BASED SYSTEM AND METHODS FOR EVALUATING TREATMENT EFFICACY TESTING WITH OBJECTIVE SIGNAL DETECTION AND EVALUATION FOR INDIVIDUAL, GROUP OR NORMATIVE ANALYSIS

(71) Applicant: Pillar Clinical Research, LLC, Richardson, TX (US)

(72) Inventors: Curtis Ponton, Santa Fe, NM (US); Brian Craig, Celeste, TX (US)

(73) Assignee: Pillar Clinical Research, LLC, Richardson, TX (US)

(21) Appl. No.: 17/643,105

(22) Filed: Dec. 7, 2021

Related U.S. Application Data
(60) Provisional application No. 63/199,109, filed on Dec. 7, 2020.

(51) Int. Cl. A61B 5/372; G16H 50/70; A61B 5/291; G16H 10/20; A61B 5/246; A61B 5/38; G16H 20/70; A61B 5/00; A61B 5/16; A61B 5/384; A61B 5/378; G16H 50/20

(52) U.S. Cl. CPC A61B 5/372; G16H 50/70; A61B 5/291; G16H 10/20; A61B 5/246; A61B 5/38; G16H 50/20; A61B 5/0006; A61B 5/16; A61B 5/4806; A61B 5/384; A61B 5/4848; A61B 5/378; G16H 20/70

(57) ABSTRACT

Systems and methods for the evaluation of clinical treatment efficacy is disclosed. The systems and methods include protocols for selection of appropriate patients/subjects for the evaluation of a specific clinical treatment. The systems and methods are based on objective measures of brain activity. The clinical treatments include pharmacological compounds in development or existing compounds approved by the appropriate regulatory authority (e.g., U.S. Federal Drug Administration), as well as transcranial magnetic or electric stimulation, including non-invasive approaches as well as grid- or depth-based electrode arrays, as well as behavioral therapies.

### CROSS-REFERENCE TO RELATED APPLICATIONS

This application claims priority benefit from U.S. Provisional Application No. 63/199,109, filed on Dec. 7, 2020. The patent application identified above is incorporated here by reference in its entirety to provide continuity of disclosure.

### FIELD OF THE INVENTION

The field of the invention relates to devices, systems, and methods that use time-series measurements of brain activity that are either spontaneous, evoked or induced by sensory stimulation either separately or combined, to evaluate the efficacy of clinically significant treatments. The field of the invention further includes investigational treatments for regulatory approval by a governing body such as the Federal Drug Administration (FDA), or other comparable foreign entity.

The field of the invention further includes to estimating minimum treatment doses for regulatory-approved treatments to assist in the determination of optimal dose levels. In particular, the field of the invention further includes detection of brain activity change related to the administration of active or placebo treatments within an individual or a group of individuals, objectively, as well against a population of normal demographically matched individuals. This includes Bayesian approaches to joint probability analysis of brain activity measured across multiple modalities.

The field of the invention also relates to neurophysiological measures of sleep. For example, schizophrenia commonly disrupts normal sleep patterns. Alterations in the normal sleep cycle include increase time to onset (latency) of sleep latency (SL), and reductions in total sleep. In addition, sleep cycle architecture is also changed with reductions non-rapid-eye movement (NREM) sleep, slow wave sleep (SWS) and rapid-eye-movement (REM) sleep latency.

### BACKGROUND OF THE INVENTION

Improvement or deterioration in the state of a patient's neuropsychiatric disorder are often drawn from clinical impressions based on observations, interviews and standardized surveys. For example, with schizophrenia, standard clinical assessment tools include the Structured Clinical Interview for DSM-IV (SCID), the Medical Informatics Information Tool (MINT), and the Positive and Negative Syndrome Scale (PANSS). While both clinical impressions and measures from standardized surveys are critical in evaluation, these assessments can be influenced by subjective variability, as well as variation in the observed behavior of a patient. Moreover, a significant increase in the placebo response has been documented, adding further variability to these measures, and contributing to the increased failure rate of clinical trials for prospective pharmaceutical or non-pharmaceutical treatments (e.g., electroconvulsive therapy, deep brain stimulation, or transcranial electric or magnetic stimulation). These factors combined make it more difficult for a pharmacological treatment (or other treatment methods) to demonstrate objective statistical improvement. Consequently, a greater proportion of investigative treatments for neuropsychiatric illness fail to achieve FDA approval status.

Neuropsychiatric illnesses reflect alterations from normal brain activity that are manifested in perceptual, emotional, motor, and/or cognitive behavior that can impact quality of life from the level of personal discomfort, to impaired social interaction and inability to maintain gainful employment through to self-inflicted and social-inflicted violence including suicide and the killing of others. Thus, in addition to the existing evaluation methods, specifically clinical interviews and standardized surveys, there has been a strong interest in the development of non-behavioral assessments of brain activity that can demonstrate objective differences between psychiatric and non-psychiatric populations. The fundamental concept in developing these objective brain-based measures is that any overt changes in symptomology associated with a specific neuropsychiatric disorder must reflect a general or specific change in underlying brain activity at some level of brain function.

Traditionally, a definition of change in evoked response activity in both basic research and clinical regulations have relied on demonstrating change or a difference by a comparison of results in one group often called a control or normal group with those from a group of interest, be it a clinical population of some kind or a treatment group, for example. For EEG and evoked potentials, such analyses have almost exclusively been performed using traditional parametric statistics between groups of subjects, with clear assumptions of normal distributions for both “real” signal in the data and the unavoidable inclusion of variability due to “noise” (defined as any activity in the continuous, epoched, or averaged EEG that is not the a priori identified brain activity “signal”. Such statistical analyses are affected by both within subject variability, but also between subject variability. Variability is reflected in differences in the magnitude of the brain activity targeted for evaluating treatment efficacy within an individual patient both within and across measurement sessions.

Variability also exists in non-targeted brain activity as well as environmental and recording/data measurement device noise that is all part of the measured brain activity. These sources of variability contribute to and increase the difficulty of obtaining a so-called treatment effect related to control vs patient comparison or pre- versus post-treatment contrast.

Consequently, devices and methods to objectively measure brain activity have been developed and applied to evaluate and measure changes in both the physical structure of the brain and functionally by assessing brain activity in a number of different ways. These methods include magnetic resonance imaging (MRI) to measure differences in the size of brain areas, functional magnetic resonance imaging (fMRI) to measure changes in regional cerebral blood flow using the so-called Bold (Blood Oxygen Level Difference) response, positron emission tomography (PET) and single position emission computed tomography (SPECT) which measures changes in regional cerebral blood flow based on the presence of an injected radioactive isotope which differential binds to areas of the brain with higher metabolic activity, as well as diffusion tensor imaging (DTI) or functional DTI which can provide measures changes in structural and functional cerebral connectivity. Obtained using a special diffusion MRI scan sequence, DTI and functional DTI, can non-invasively map the diffusion process of molecules, primarily water, in biological tissue including the brain. With tractography, DTI data can provide statistical estimates of fiber tract projections (brain white matter axons) between cortical and sub-cortical areas.

Studies show that it may further be possible to evaluate the functional state of these fiber tract projections associated with near infrared spectroscopy (NIRs) or imaging (NIRI) which is used to measure changes in the optical properties of tissue simultaneously from multiple measurement points. When applied to neuroimaging, activity-dependent changes in local superficial cerebral blood flow can be evaluated using time-varying maps displayed as a function of sensor position on the scalp or projected onto an MRI-based average or individual reconstruction of the cortical surface.

Paradigms assessing changes in brain activity between neuropsychiatric populations and “normal” non-psychiatric populations identify differences in spontaneous and evoked brain activity employ anywhere from zero events (spontaneous brain activity) to multiple unique events (evoked brain activity). For both spontaneous and evoked brain activity paradigms, EEG and MEG measurements begin with the acquisition of continuous ongoing brain activity.

Many of these measures of structural and/or functional brain activity have demonstrated their value identifying differences that can be objectively measured between normal and neuro-psychiatric populations. However, in the case of developing metrics for evaluating acute and long-term effects of treatments, such as drug compounds, they are generally insensitive in showing more short-term effects on the order of minutes, hours, or days. The exceptions to this are PET and fMRI, application of which is limited by widespread availability, cost, and in the case of PET (or SPECT), introduction of a radioactive isotope, which can limit repeat testing, particularly in pediatric populations.

All the above methods have limitations in terms of acute changes in short periods of time in that the methods are based on brain activity data integrated over much longer time periods to demonstrate change. For MRI and DTI, structural changes occur on the time scale of hours to days to months to years. For fMRI, NIRs or NIRI, and PET, the time scale is much better but remains on the order seconds to minutes.

Many functional brain processes from simple perception to more advanced cognitive process occur on very short time scales, from milliseconds to seconds. This has been demonstrated using electroencephalography (EEG) and magnetoencephalography (MEG), and an emerging homologue to MEG, optically pumped magnetometers (OPMs). Potentially, functional tractography based diffusion MRI (fDTI) may also provide very time sensitive approach to evaluating change in the activation of white matter pathways in the brain as well.

For the measurement of EEG, electrodes are placed on the scalp to record electrical activity generated by brain that is propagated through the volume separating the brain and the scalp, which includes weakly electrically conductive cerebral spinal fluid, the meninges tissue encapsulating the brain primarily the dura matter and the arachnoid layer, as well as the skull and the skin.

For MEG, the magnetic counterpart of the electrical field passes transparently through the tissues and fluids surrounding the brain and is measured with sensors known as superconducting quantum interference devices (SQUIDS), bathed liquid helium, which thus allow measurement of the extremely minute time-varying magnetic fields generated by electro-chemical activation of neurons, which are measured by EEG Both EEG and MEG methods provide extremely high temporal resolution on the order of milliseconds.

For all sensory modalities and for so-called resting state brain activity, there are changes in spontaneous activity measurements of the EEG time waveform, and the corresponding spectral structure of this activity. Moreover, there are regional differences in brain activity associated with each of the sensory modalities, as well as with more complex cognitive processing including language perception and production and memory, as a very brief list of examples.

For schizophrenics, abnormalities in sleep have been associated with memory consolidation deficits. Sleep abnormalities are common in other neuropsychiatric disorders, including schizophrenia, depression, and anxiety, and Alzheimer's disease as few examples.

Others have suggested that sleep disturbances are not just a symptom of psychopathology but reflects a causal relationship between sleep disturbance and neuropsychiatric disorders. The suggested causal links include the co-existence of sleep disturbance with emotional lability, genetic correlations with neuropsychiatric disorders, and specific dysfunctions associated with dopaminergic and serotonergic neurotransmitter systems.

Among the earliest measurable brain responses is the auditory brainstem response (ABR), occurring in the first 10 ms after the presentation of an auditory stimulus, either in the form of a short DC offset (auditory click), tone, or speech stimulus. Among its many applications, the ABR is clinically used to objectively evaluate hearing loss and for the detection of acoustic neuromas.

Visual and auditory evoked potentials have been used extensively for a number of applications including brain development and aging, language, memory, and attentional processes under a number of conditions in normal populations. For neuropsychiatric populations, a number of the methodologies used to increase our understanding of normal brain function from perception through to cognition have demonstrated differences in brain activity that distinguish neuropsychiatric patients from the normal population. In addition, some studies have been published demonstrating that EEG and/or MEG can be used to demonstrate differences between groups in brain activity following drug treatment between groups of normal and patient populations.

Traditionally, these methods have relied on the visual identification of peaks and troughs in the EEG waveforms, which in temporal proximity, are described as components of the brain's event related potentials (ERPs) or evoked potentials (EPs) activity. Unfortunately, the identification of peaks and troughs, either visually (manually examining each waveform) or automatically, does not effectively account for the background “noise” in the EEG, which is the residual brain activity or environmental noise, that is not associated with the presentation of sensory stimulus event. This leads to increased variability both within and across individuals which, in general restricts analysis and statistics to be based on group versus individual subject data.

A number of techniques have been used to objectively measure changes in brain activity across the realm of the methods described above. For fMRI and PET, methods known as statistical probability mapping (SPM) are the accepted methodology for objectively measures ongoing local changes in brain activity. Typically, these approaches require a task change (simplest being no task to task). For EEG and MEG, the presence of brain activity that is either spontaneous or evoked by a sensory event incorporates both background brain signal and muscle noise, as well as the brain response evoked by a repeated presentation of an auditory, visual, or other sensory event. Because the magnitude of the brain responses smaller than that of the ongoing and often unrelated background brain activity, eliminating noise is important.

One of the first methods for objectively evaluating the presence of a change in evoked activity, for example, was for the auditory brainstem response (ABR). The method used estimates of variability in the EEG measured at the same single time point in an each evoked potential epoch against the variability of a number of consecutive time points defining the ABR waveform to calculate the so-called Fsp value or single point F-ratio, where the denominator is based on the variance using the single time point as an estimated of the noise, while the numerator is based on the variance across the series of time points encompassing the ABR.

The method was specific to the ABR as the assumption of stationarity of the neural response was largely fulfilled. As such, a signal-to-noise ratio could be estimate in which the denominator of the ratio is based on variability in measured EEG brain voltage at a single arbitrary time point in the ABR time waveform. Variability in measured brain activity across a consecutive series of digitized points encompassing the ABR waveform constitutes the numerator. Of course, by definition, the numerator not only includes the brain signal response to the auditory stimulus, but it also includes the variance of the single time estimate for each time the auditory stimulus was presented. Thus, the ratio contained measured brain signal plus measured brain noise in the numerator, compared to measured brain noise in the denominator, in effect, an approximation of the typical statistical F-ratio used in statistics.

Prior art methods have also been developed related to the measurement of cortical evoked potentials. These methods differ from those used to measure the auditory brainstem response in that they do not include the assumption of stationarity of the neural response. Unlike the ABR, the neural activity of thalamocortical or cortical evoked potentials was not justified, as thalamocortical and cortical potentials, unlike the ABR, are affected by many factors such as, but not restricted to, state of arousal, in which sleep would attenuate or prolong the latencies of the measured evoked potential components. In contrast, the ABR neural generators were essentially unaffected by sleep and only manifest significant changes to alcohol and narcotic drugs.

Methods have also been developed that evaluate the current noise content in the averaged EEG response against a pre-set threshold, which represents a stopping criterion for the hearing loss evaluation.

Other prior art methods, known as “t-tests”, compare peak latencies and amplitudes of evoked potential components (defined as a peak and following trough in the EEG waveform). These methods are time intensive when performed manually and heavily impacted by residual unaverage noise in the evoked potential signal. Hence, this method also fails to provide for the detection of excessive noise in relation to non-physiological noise sources or the use of normative data.

Other prior art methods have provided a topographic analysis of EEG voltage map which incorporates into its estimate of statistical significance both the noise of and the signal of evoked thalamocortical and cortical evoked brain activity.

The prior art has also developed Statistical Probability Mapping (SPM) to address the immense number of sampling points that could reflect “real” changes in brain activity found with PET, SPECT, and fMRI, against random measurement changes due to the device or other parameters not related to the intended manipulation of brain activity. These methods provide approaches to determine on a single subject basis, whether a significant change in brain activity has occurred between states (perceptually or cognitively active or resting states), as well as statistical maps identifying local brain regions of change. SPM reflects a different approach but similar in intent to provide a statistical probabilistic estimate of change in brain state, even at the level of the patient.

Traditional parametric approaches to statistical testing of changes or differences in brain activity, apply the common practice of manually, or with some devices and systems, or automatically selecting a peak (maximum positive amplitude) and trough (maximum negative amplitude) of an EEG or MEG component, such as an auditory P50 or N100. In the frequency domain, a peak at a specific frequency or group of frequencies might be selected.

For example, in an evoked response the calculation of the signal-to-noise (SNR) is (Signal+Noise)/Noise. In any average of brain activity signal (the brain response) can never be entirely separated by noise (the portion of the measured brain activity that reflects unrelated back brain activity, environment noise, recording equipment noise, etc.).

For an SNR of 10, calculation of the SNR=((1+9)/1), or (10/1). where 9 represents the signal and 1 represents the residual noise, provides the SNR of 10. In in this case, noise represents roughly 10% of the measured peak or trough value. For a SNR=5=(1+4)/1, or (5/1), noise may represent as much as 25% of the measured peak and trough amplitude. Thus, traditional measurements, if anything, conflate and thereby enhance the size of a “real” evoked response component with residual unrelated random brain noise that is not entirely minimized by signal averaging. The problem becomes worse across evoked or induced measures of brain activity measured across multiple subjects.

Traditionally, evoked or induced measures of brain activity are recorded to a fixed number of stimulus presentations, with no guarantee across measurements from a single individual or across individuals, that there is consistency in the signal-to-noise ratio.

### SUMMARY OF THE INVENTION

The present invention represents a major advance in the state-of-the-art because it allows for objective measurements that included background “noise” as part of the calculation for estimating significant change in pre-/post-neuropsychiatric treatment conditions at both the group level and at the level of the individual patient. Moreover, precise selection of stimulus modalities and paradigms can allow multiple measures that can be applied in a joint probability model, incorporating Bayesian principles to provide more robust measures of outcome. Joint probability models allow increased statistical confidence by, in principle allowing a combination of at least partly or entirely independent statistical measures, into a single probability estimate.

As an example, the probability of an object being an edible fruit such as an orange, is greatly enhanced by having measures of color, taste, smells, and shape—as in if looks like and orange (round and orange in color) and tastes and smells like and orange, it is much more likely to be an orange than if we only know the shape is round. Further, if we have prior knowledge, such as the “object” grew on a tree, this excludes other round objects such as tennis balls, potatoes, and other non-round of spherical objects that do not grow on trees. Moreover, the analysis can be performed not only on group comparison, but also on and individual basis.

The present invention provides an objective analysis for deriving statistically significant estimates of change thus allow an individualized medicine approach applicable to both the development of treatments such as drug compounds, with the potential to provide a metric in assessing treatment efficacy (i.e., drug dosage) for FDA approved (drug) treatments. Equally important, is that brain-based objective analyses such as that of the invention, provides evidence of unintended side effects of medications under development or those that are already approved. Unintended side effects may be reflected in brain-based measures of activity from the central or the autonomic nervous systems.

The present invention provides devices and methods for use in objectively identifying changes in brain activity related to a clinical treatment based on spontaneous brain activity or brain activity evoked or induced by stimulation from any sensory modality. The change in treatment-related brain activity can be caused by active drug compounds under development, active approved drugs, active magnetic or electric stimulation, active behavioral treatment, active gene therapy as well as appropriate placebo treatments.

The invention is targeted toward human investigation but is also applicable to non-human studies of treatment effects. The invention is directed to systems, devices and methods that are capable of detecting whether there is a significant change in brain activity that distinguishes between a pre- and post-treatment state based on the use of individual subject data, group data, or comparing individual or group data with normative data from non-neuropsychiatric populations.

The present invention is also directed to devices, systems, and methods capable of detecting and measuring noise levels of continuously measured brain activity, or time-series epoched measures of brain activity and the associated averages of that activity, in the time, frequency, or spatial domains. The said targeted brain measured across a time epoch is defined as a period of spontaneous or evoked brain activity of arbitrary duration sufficient to capture the brain activity of interest.

The invention also includes a sorting algorithm that ranks epochs based on high to low noise levels, with a functionality of automatically rejecting epochs that exceed a certain noise range (high and/or low) or those that show a specified percentage/standard deviation of the average noise level of all epochs.

Additionally, this invention allows for online, near-real time estimation of signal-to-noise ratios to enhance the signal-to-noise quality of the averaged response from all accepted individual epochs.

The system described as part of the invention, acquires continuous spontaneous and/or evoked and or induced time-series epochs of brain activity measured separately or as part of a continuous data acquisition protocol. This system includes appropriate sensors, amplifiers, and signal conditioners such as filters, as well as computer hardware, software, and digital data storage devices appropriate for measured form of brain activity measurement (e.g., EEG vs fMRI vs PET vs NIRS). The measured brain activity may be spontaneous, directed or non-directed mentation, or it may be in response to one or to a plurality of sensory stimuli collected in pre- and in post-treatment (active or placebo) conditions. Each time-epoch extracted from a continuous recording brain activity contains single or multiple spontaneous or stimulus-evoked/induced time samples at as few one sensor location proximal to the head, and therefore the brain, to a plurality of locations distributed on or near the head, providing a more complete evaluation of underlying brain activity. The duration of the sampled time-series epoch is of a sufficient duration to capture the targeted brain activity of interest. Alternatively, individual epochs extracted from a measurement of spontaneous brain activity can be collected at pre- and post-treatment time points. These epochs are subjected to a novel statistical analysis which clearly indicate the presence or absence of change.

As part of the invention, spontaneous and evoked brain responses and are sorted and averaged based on collection time points determined by pre- vs post-treatment administration (with the understanding that the post-treatment measurement may be obtained from an individual on an active treatment agent or an inert, non-active placebo treatment). The epoch averages, when appropriate, are calculated in the time and/or frequency domain. Comparisons are then based on a point-by-point basis for the duration of the epoch. The epoch duration for spontaneous activity may depend on the nature of a passive versus active task, but not restricted to mentally relaxing or performing math calculations, or any other activity. For stimulus event evoked epochs, the epoch duration may vary depending on the modality of stimulus presentation and, in the specific case of evoked activity, the time (latency) of the evoked brain activity component of interest.

Traditionally, a definition of change in brain activity in both basic research and clinical regulations has relied on demonstrating a difference by comparison of results in one group often called a control or normal group with those from a targeted group of interest, be it a clinical population of some kind or a treatment group. For the preferred embodiment of this invention using EEG or MEG, analyses of spontaneous, evoked, or induced brain activity in the time or frequency domain, been performed using parametric statistics between groups of subjects, with clear assumptions of a normal distributions for both “real” signal in the data and the unavoidable inclusion of variability due to “noise”. Such statistical analyses are affected by both within subject variability, but also between subject variability.

Variability can reflect differences in both the magnitude of the actual spontaneous or evoked brain response but also in non-task related background brain activity as well as environmental and recording/data measurement device noise that is all part of the measured brain activity. These sources of variability contribute to and increase the difficulty of obtaining a so-called treatment effect related to control vs patient comparison or pre- versus post-treatment contrast.

A goal of this invention is to minimize both within and across subject variability for the evaluation of treatment efficacy. Another goal is to provide estimates of treatment efficacy at the level of the individual patient, thus following a model of data driven, individualized medicine.

In additional to traditional parametric statistical analysis, the invention includes non-parametric and Bayesian statistical analysis approaches that are applied for both individual and group level comparisons as well as joint-probability analysis, when it is appropriate to combined different measures of brain activity.

The invention includes non-parametric permutation/randomization methods that are distribution free, producing exact probability estimates. This mitigates the need to rely on parametric statistics which make use of probability calculation table assuming normal distributions of the data included for analysis.

As a primary mode of analysis, this invention adopts as the preferred statistical analysis an approach that inherently assumes the presence of noise as part of the recorded brain activity in all calculations of significance. At its simplest, and using EEG as an example, this test for significance is a determination of whether or not the targeted brain response (a particular component or peak, in the time domain, or a particular frequency or set of frequencies in the spectral domain) is greater than background “noise” activity in one condition or state. Further tests determine whether or not a change in state (e.g., pre- versus post-treatment) results in a significant change in brain state, above and beyond noise in the brain measurement, un-associated with either state.

For single subject and group analysis, this non-parametric-based analysis starts with the assumption of the null-hypothesis; simply put, the treatment (active treatment or placebo) has no effect. For the single subject, this is tested by determining the size of the difference obtained between spontaneous or evoked brain responses (in the time or in the frequency domain) based on averaging of epochs to form an epoch average for the pre-treatment state, with another epoch average for the post treatment state. This difference, regardless of magnitude becomes the “obtained difference”.

Then, the method effectively “blinds” itself as to whether a time-series epoch is obtained from a pre- vs a post-treatment data acquisition session, by randomly substituting a single epoch from the pre-treatment series of epochs with a single epoch from the post-treatment series of epochs. A set of epoch averages is then obtained for both the substituted pre-treatment epochs and the substituted post-treatment epochs. The difference between the substituted pre- and post-treatment averages is then obtained as a substitution obtained difference. The substitution obtained difference is then compared to the “obtained difference” to determine a percent change.

Because of the typically low SNR associated with sensory event stimulation, with EEG for example, averaged evoked or induced brain activity is typically based on tens, to hundreds, to thousands of individual time-series epochs. The total number of possible combination increases exponentially as the numbers of epochs increase. For a simple mathematical example. If an unrealistically low number of 10 is used for both the pre- and post-treatment number of epochs, then the total number of combinations is based on the following formula:

$${C}_{( n , r )} = \frac{n !}{r ! ( n - r ) !}$$

Where:
C=the number of combinations;
n=the total number of epochs in the pre-treatment and post-treatment groups; and,
r=the number of epochs in the pre-treatment group.

In our example, number of possible combinations is 20!/(10×(20−10)!)=184,756, resulting in a total of combinations. While an exhaustive calculation of all possible combinations is prohibitively time consuming, in the invention, the user has the option of specifying that a random subset of combinations be calculated, which can be analyzed very rapidly, on the order of seconds to minutes.

For EEG, analyses can be performed on all digitized epoch time points measurement of the brain activity from multiple sensor channels (or regions of interest) or with as few as a single sensor channel. Results can provide a time (or frequency) point-by-point probability estimate of significant change across all sensors. Optionally, a subset of sensors (down to a single sensor) or a region of interest of sensors (e.g., for EEG, midline electrodes) may be a priori selected for analysis.

Similar calculations can be performed on group data, in which obtained averages of brain activity for individual subjects from pre- and post-treatment groups can be compared with substituted group averages and tested against the null hypothesis. In each case, a final calculation is made of how often the obtained difference between pre-/post-treatment averages is greater than differences between substituted pre-/post treatment averages. This approach generates exact probability values as a function of time (or frequency in the spectral domain) based on the number of substitutions for each contrast.

In the preferred embodiment, the preferred number of combinations will be automatically calculated with a minimum number set at 1000 combinations, providing a maximum probability of p=1/1000 or p=0.001.

The above statistical analysis can be performed for any epoched-based averaged brain activity, in either the time or in the frequency domain. More than one measure of averaged brain activity employing different stimulus presentation paradigms and different sensory modalities may be impacted with many neuropsychiatric disorders.

In the preferred embodiment, at least one and perhaps multiple measures of averaged time or frequency domain brain activity may be employed to determine if changes existed in the pre- and post-treatment states.

For example, for schizophrenia, differences in EEG brain activity exist between non-schizophrenic control populations for spontaneous as well auditory, visual, transcranial electric and transcranial magnetic stimulation. Within or across sensory modalities, measured brain activity may reflect a similar stage of cortical processing.

As another example, differences in EEG brain activity exist at the perceptual cortical level where event detection occurs, or at higher level of cognitive analysis including discrimination between different sensory events from within the same modality or detection of a specific target (e.g. tone of a different frequency, visual object of a different shape, color or size) or advanced levels of processing such as discrimination between different faces based on some characteristic such as familiarity, race, age, or some other factor.

The invention may be employed independently for each measure of brain activity or may be combined using a joint probability analysis. Joint probability analyses can be employed to increase power across a series of tests where it may be difficult to achieve tradition statistical levels of significance, for each p<0.05, or 95% confidence of rejecting the null hypothesis.

For example, if it can be demonstrated that those different measures of brain activity are entirely independent, simple joint probability models can be employed. As an example, lets refer to the identification of an object as an orange. For simplification, three modalities or features are employed; shape (the individual was pick one of ten shapes/sizes), color (from a list of ten colors), and taste (one of ten different fruit taste options).

In a simple model, the chances of an individual randomly guessing at each feature and selecting the features that defined an orange would be a 10% chance of being correct for each of the features, which statistically, there would be a 0.1×0.1×0.1 or a 1 in 1000 chance of being correct by chance. Expressed as a percentage, this would be a 0.001% chance of randomly selecting the features that would uniquely define and orange in this simplified example. For testing whether there is a significant change in brain activity, we will use an example of schizophrenia.

There are at least three different measures of EEG-based brain activity that show robust changes between schizophrenic and non-schizophrenic subject groups. First, there are measures of passively (non-attentively) measured brain activity that for EEG or labeled P50, suggested to be a measure of early attentional gating, which is a component define by positive/negative peak structure at 50 ms in the averaged auditory evoked brain response.

Second, there are measures of the mismatch negativity (or MMN), which reflect an automatic detection of novelty. The MMN is defined as an added negative component between 100-300 ms in latency in the average auditory evoked brain response in response to a physically different/deviant sound (e.g., louder, longer, different frequency) in response to infrequently presented sound embedded in a presentation sequence of a physically different frequently presented sound.

Third is the P300 response which discriminates between schizophrenic and non-schizophrenic populations. The P300 response requires actively attending to a stimulus sequence. This component of evoked EEG is positive waveform at 300 ms in average the evoked brain activity and can be evoked by stimuli from any sensory modality. For the P300, the participant actively attends to the presentation sequence and makes a response such as a button press, when the specified infrequently occurring target/deviant stimulus occurs. For each of these three measures of brain activity, a measure of statistical change with an assigned exact probability can be obtained. Depending on the SNR levels in the data, no single test, or conversely all three tests may provide an outcome with a 95% confidence, p<0.05 value. However, assuming independence of the three-measure joint probability can be applied to gain more power.

For example, if it is assumed that each measure has a probability of p=0.2. A joint probability would then consist of 0.2×0.2×0.2=0.008, below the commonly accepted probability of p<0.05.

Unfortunately, complete independence of the three measures cannot always (or perhaps ever) be assumed. Therefore, the invention also provides more advanced approaches to non-independent or partially dependent measures of brain activity. For example, the following additive model for combining probabilities may be employed:

p_{c}=Sⁿ/n!

Where:
p_{c}=combined probability value;
S=sum of the probability values;
n=number of probability values; and,
n!=the factorial value of the number of probability values.

Thus, for the previous example, S=(0.2+0.02+0.2)=0.6, 0.6, n=3, n!=6

Or p_{c}=0.6³/3!; or 0.007

There are a number of alternative non-parametric methods for calculating combined or joint probabilities for neuroimaging data that may be employed in the invention. In addition, application of Bayesian methods, that account for possible contributions of covariance across brain activity measures and adjust joint probabilities appropriately to more conservative values, may be applied when necessary.

The invention's inclusion of a Bayesian statistical approach allows what is “prior knowledge” to be factored into the statistical estimate. Measures of brain activity as biomarkers are considered more objective, as there is much less human (clinician and patient) contribution to the final estimate of determining whether there is a change in brain activity related to a pre-/post drug state. However, measures of brain activity, from behavioral activities associated with a neuropsychiatric condition can and do provide value information on the existence and severity of a disorder. Thus, a clinical opinion provides important “prior knowledge”.

For example, with schizophrenia, a clinical opinion is often followed up by administration of the PANSS, MINT or SCID scales, which can provide more information regarding the severity of the disorder. While there are concerns regarding the variability and objectivity of PANSS scores, this information still provides “prior knowledge” that can be included if available as part of an overall assessment, including the objective brain neurophysiological brain measures outlined in this invention.

Given the importance of sleep for neuropsychiatric/mental health, the invention also provides for the collection and scoring of sleep data as a critical component in the assessment of drug compound efficiency. Results of the sleep studies will be an independent measure of drug efficacy. However, since sleep could impact brain activity in the awake state, this data is used to optimize the time of day for acquiring brain-activity in the awake state. Thus, the sleep data is included in the joint-probability analysis of spontaneous, evoked, and induced brain activity recorded while awake.

The invention also includes a device for continuous, ambulatory monitoring sleep/state of arousal. By selecting test times at optimal awake/state of arousal intervals, at least some of the “noise” in targeted measures of brain activity should be minimized. This likely applies to a lessor or greater extent to all measurements of brain activity regardless of method (e.g., EEG vs MEG vs fMRI vs PET vs NIRs). Background EEG, unrelated to the actual evoked brain response, varies greatly from individual to individual. Consequently, assumptions regarding normal distributions, upon which traditional parametric statistics are based, are rarely tested, and may often not be unjustified.

In addition, the data obtained from the ambulatory sleep monitoring/state of arousal device will be used to identify optimal times to measure the spontaneous, evoked or induced brain activity previously mentioned.

### BRIEF DESCRIPTION OF THE DRAWINGS

In the detailed description of the preferred embodiments presented below, reference is made to the accompanying drawings.

FIG. 1 is a block diagram of an EEG brain activity measurement system.

FIG. 2 is a network diagram of an EEG brain activity measurement system.

FIG. 3 is a diagram of an EEG device and headset.

FIG. 4 is a diagram for a method of an EEG brain activity measurement system.

FIG. 5A is a flowchart of a preferred method for determining percentage change between pre- and post-treatment.

FIGS. 5B and 5C is a flowchart of a preferred method of a substitution analysis.

FIG. 5D is a flowchart of a preferred method of combined probability analysis.

FIG. 5E is a flowchart of a preferred method of Bayesian probability analysis.

FIG. 6A is an illustration of brain activity changes for a patient from a double-blind drug trial at day 4 and day 14 following assignment to the active drug group indicating the effectiveness of the invention.

FIG. 6B is an illustration of brain activity changes for a patient from a double-blind drug trial at day 0 and day 14 following assignment to the active drug group indicating the effectiveness of the invention.

FIG. 7 is a summary of results for changes in P50 attenuation and PANSS scores as a function time, across day 0, day 4, and day 14 indicating the effectiveness of the invention.

FIG. 8A is a correlation analysis showing the relationship between auditory P50 measures of attentional gating and PANSS scores for day 4 indicating the effectiveness of the invention.

FIG. 8B is a correlation analysis showing the relationship between auditory P50 measures of attentional gating and PANSS scores for day 14 indicating the effectiveness of the invention.

FIG. 8C is a correlation analysis showing the relationship between auditory P50 measures of attentional gating and PANSS scores indicating the effectiveness of the invention.

FIG. 9 is an averaged evoked EEG auditory P50 brain activity superimposed on the 80+ individual time-series epochs included in each average at Day 4 and Day 14 of drug treatment.

### DETAILED DESCRIPTION OF THE INVENTION

In the description that follows, like parts are marked throughout the specification and figures with the same numerals, respectively. The figures are not necessarily drawn to scale and may be shown in exaggerated or generalized form in the interest of clarity and conciseness. All tolerances are plus or minus 20% unless otherwise specified.

Systems and methods for the evaluation of clinical treatment efficacy is disclosed. The systems and methods include protocols for selection of appropriate patients/subjects for the evaluation of a specific clinical treatment. The systems and methods are based on objective measures of brain activity. The clinical treatments include pharmacological compounds in development or existing compounds approved by the appropriate regulatory authority (e.g., U.S. Federal Drug Administration), as well as transcranial magnetic or electric stimulation, including non-invasive approaches as well as grid- or depth-based electrode arrays, as well as behavioral therapies.

The systems and methods provide use and analysis of a plurality of spontaneous, sensory stimulus evoked and induced measures of brain activity to determine, within and between individuals and across groups, whether there are objectively measurable changes in brain activity between conditions of pre- and post-treatment, as well as active or placebo treatment. To facilitate efficient differentiation of brain activity changes from background environmental and other noise sources in a measurement, statistical measures are employed to differentiate environmental and other non-brain sources of activity from potential changes in said brain activity associated primarily with the pre- versus post-treatment (active of placebo) state.

Also disclosed is a function to perform comparisons for pre- and post-treatment states with a normative database of brain activity measured by a comparable measurement system and brain activation paradigm. The invention thus includes the functionality of determining within a single measurement, whether brain activity falls within or outside normal parameters for a specified treatment and associated test condition, thus providing a means by determining if an individual is appropriate for inclusion in a treatment study.

Definition of Targeted Populations: Human and non-human species.

Definition of Treatments: Including but not restricted to pharmaceutic compounds in development, drugs with regulatory approval, all forms of electric and magnetic brain stimulation, including but not restricted to deep brain stimulation, electro-convulsive therapy, transcranial repetitive (or non-repetitive) DC or AC electrical scalp stimulation and transcranial magnetic stimulation, behavioral treatment, genetic therapy/treatment and any combination of treatments.

Definition of Disorders: Including but not restricted to the range of (neuro)psychiatric disorders including, schizophrenia, Alzheimer's disease, autism, attention deficit hyperactivity disorder, obsessive-compulsive disorder, and depression of all varieties, including bipolar disorder, as well as pain, either real or psycho-somatic. Other brain-based disorders including Parkinson's disease or addictive disorders are also included. This patent is intended to include the evaluation of treatment efficacy for other brain-based disorders.

Definition of Brain Based Disorders: Disorders that affect the brain as well as the nerves found throughout the human body and the spinal cord. These can include but are not restricted to Alzheimer's Disease, dementias, brain cancer, Epilepsy and other seizure disorders, mental disorders, Parkinson's and other movement disorders, stroke and Transient Ischemic Attack (TIA).

Definition of Central Nervous System (CNS) Brain Activity Measurements: Including but not restricted to, functional magnetic resonance imaging (fMRI) to measure changes in regional cerebral blood flow using the so-called Bold (Blood Oxygen Level Difference) response, positron emission tomography (PET) and single position emission computed tomography (SPECT) which measures changes in regional cerebral blood flow based on the presence of an injected radioactive isotope which differential binds to areas of the brain with higher metabolic activity, diffusion tensor imaging (DTI) or functional DTI (fDTI). Magnetic Resonance Spectroscopy (MRS) which can provide measures of changes in specific neurotransmitters in the brain, Near Infrared Spectroscopy or Imaging (NIRs or NIRI), which provides measures of changes in local regional cerebral blood flow with a laser sensor/receiver placed on the scalp, electroencephalography (surface, cortical grid or depth electrodes) which provides a high temporal resolution measure of electrical activity from the brain measurable in both human and non-human species magnetoencephalography which provides a high temporal resolution measures of the magnetic component of the electrical field activity recorded by sensors located above the from the brain measurable in both human and non-human, including measurements using optically-pumped magnetometers (OPMs).

Measures of these forms of brain activity may be spontaneous with or without directed task or evoked where activity is task-related and is more closely time-lock and in the case of EEG/MEG, phase-locked to the presentation of events in the tasks, or induced, where brain activity is roughly time-locked to presentation of task/stimuli, but in the case of EEG/MEG, may not be phase locked. This includes but is not restricted to the following paradigms used to generate evoked or induced brain such as the pre-pulse inhibition task, P50 suppression task, passive or active oddball presentation tasks, continuous cognitive processing tasks, or higher cognitive tasks including facial identification and emotional processing.

Definition of Autonomic Nervous System (ANS) Measurements: Including but not restricted to heart rate and heart rate variability as measured by electric, magnetic, or photo-reflective (laser-based) technologies. Another example is gate stability as measure by actigraphy technologies. Measures of these forms of brain activity may be spontaneous with or without directed task or evoked where activity is task-related and is more closely time-locked and in the case of EEG/MEG, phase-locked to the presentation of events in the tasks, or induced, where brain activity is roughly time-locked to presentation of task/stimuli, but in the case of EEG/MEG, may not be phase-locked.

Definition of PANSS: PANSS is a medical scale used for measuring symptom severity of schizophrenia and is determined through a brief interview by a clinician who gives a score from 30 to 120.

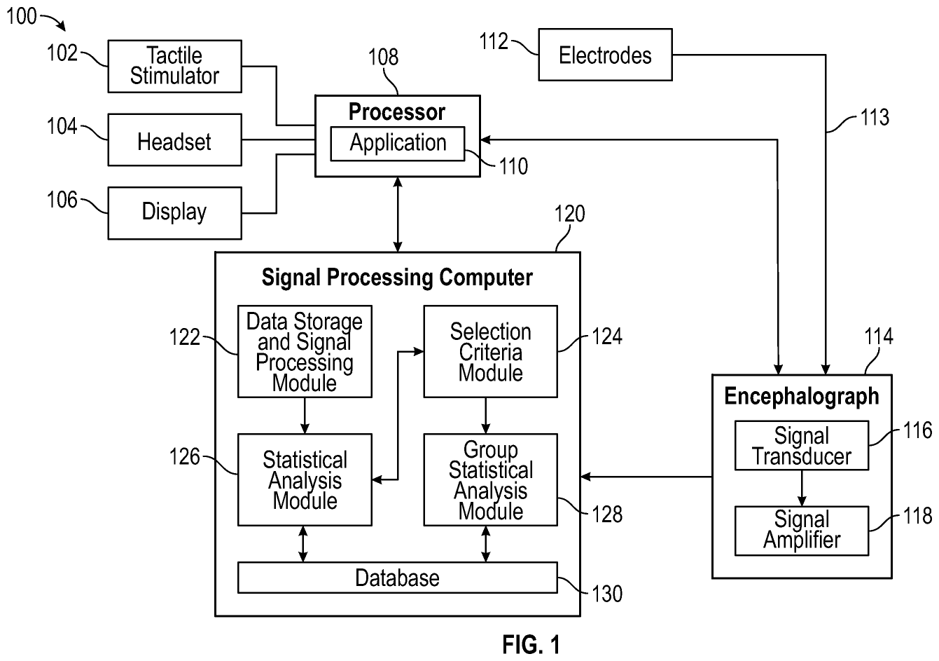

Referring then to FIG. 1, a preferred embodiment of system 100 for objectively evaluating brain activity change for neuropsychiatric disorders pre- and post-treatment will be further described.

Tactile stimulator 102, headset 104, display 106 are operatively connected to processor 108. Tactile stimulator 102 presents a tactile stimulus to the patient. In a preferred embodiment, the tactile stimulation is a Galileo Tactile Stimulus System available from Brainbox Ltd of Cardiff, United Kingdom. Headset 104 presents an audio stimulus to the patient. In a preferred embodiment, the headset is a SmartingPRO available from mBrainTrain, LLC of Belgrade, Serbia. In another preferred embodiment, the headset is a Quick-Cap electrode array available from Compumedics Limited of Abbotsford Victoria, Australia. Display 106 presents a video stimulus. In a preferred embodiment, the display is an LCD monitor.

Processor 108 includes application 110 stored in suitable memory. Application 110 controls the activation of encephalograph 114 and generation of signals which activate the tactile stimulator, the headset and presents images on the display, as will be further described. In a preferred embodiment, processor 108 is provided in the Grael 4K PSG:EEG Amplifier and Recorder, available from Compumedics Limited of Abbotsford Victoria, Australia

Processor 108 is operatively connected to encephalograph 114 and signal processing computer 120, both of which will be further described. Processor 108 through application 110 notifies the signal processing computer that a test has been initiated. Likewise, processor 108, through application 110 activates encephalograph 114 when a test is initiated.

Encephalograph 114 receives analog signals from electrodes 112 attached to a patient. Encephalograph 114 includes signal transducer 116 and signal amplifier 118. Signal transducer 116 converts the analog signals into usable electrical signals. Signal transducer 116 may take capacitive, inductive or resistive form depending on modality. Signal transducer 116 is connected to signal amplifier 118. Signal amplifier 118 converts the electrical signals from signal transducer 116 into more discernible signals for further digital processing. Signal amplifier 118 is a differential amplifier that electronically stabilizes and amplifies the signal. Typically, a differential amplifier is provided for each pair of electrodes. Signal amplifier 118 can include a buffer amplifier, which stabilizes and amplifies by a factor of five (5) to ten (10) and a pre-amplifier, which filters and amplifies by a factor of ten (10) to a hundred (100). In a preferred embodiment, encephalograph 114 is provided in the Grael 4K PSG:EEG Amplifier and Recorder, available from Compumedics Limited.

Electrodes 112 are connected to encephalograph 114 through connecting wires 113. Electrodes 112 can be surface or needles electrodes or combined into an electrode cap. In a preferred embodiment, adhesive tape is used to attach electrodes 112 to a patient. Electrolyte gel or paste is applied to the skin to help form a conductive bridge between the skin of a patient and electrodes 112 to allow better signal transmission. Electrodes 112 can be disposable, such as tab, ring, or bar electrodes, or alternatively, electrodes 112 can be reusable disc or finger electrodes. Electrodes 112 are placed at multiple spatial locations on the scalp using the international 10/20 system and correspond to the underlying area of the brain. In a preferred embodiment, electrodes 112, are included in a wearable thirty-two (32) channel EEG headset, for brain computer interface applications, sold under the tradename Quick-Cap available from Compumedics Limited. In another preferred embodiment, electrodes 112 are included in a wearable eight (8) channel EEG headset sold under the tradename Unicorn Hybrid Black, available from g.tec neurotechnology GmbH, of Schiedlberg, Austria.

Encephalograph 114 is connected to signal processing computer 120. Signal processing computer 120 includes data storage and signal processing module 122, selection criteria module 124, statistical analysis module 126, group statistical analysis module 128, and database 130. Data storage and signal processing module 122 is responsible for monitoring raw electrical signals and for conversion into distinct frequency bands alpha, beta, theta and gamma, using a fast Fourier or wavelet transform and then storing the resulting frequency charts in files.

Statistical analysis module 126 is responsible for conducting analysis of the stored frequency charts, for individual patients, as will be further described. Group statistical analysis module 128 is responsible for conducting analysis of the stored time series or frequency charts, for patient groups, as will be further described. Selection criteria module 124 is responsible for screening the frequency charts of individual patients for inclusion in group studies according to a predetermined set of criteria, such as age, disease, date or signal modalities. Once the time series or frequency charts are grouped for analysis, they are stored in database 130. In a preferred embodiment, signal processing computer 120 takes a form of the Unicorn Suite Hybrid Black software environment, including a Unicorn Recorder, Unicorn CAPI, for communication to processor 108, all available from g.tec neurotechnology. In an alternate embodiment, the signal processing computer employs the Curry8® signal processing software available from Compumedics Neuroscan of Abbotsford, Victoria, Australia. Database 130 is preferably an Oracle database, including appropriate APIs for rapid query response, from signal processing computer 120.

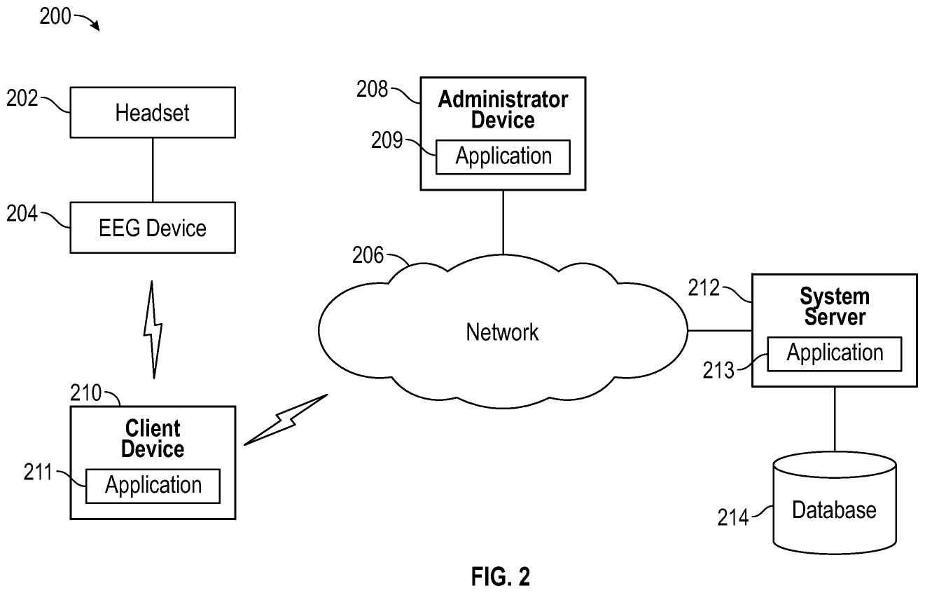

Referring then to FIG. 2, a preferred embodiment of system 200 for ambulatory EEG brain activity measurement will be further described.

System 200 is comprised of headset 202 and EEG device 204 which are wirelessly connected to client device 210. Client device 210 is a smart device such as a computer, tablet, or cell phone. Application 211 is resident on client device 210 is responsible for presenting stimulus and coordinating communication between administrator device 208 and EEG device 204, as will be further described. Client device 210 is wirelessly connected to network 206, such as the internet.

The system is further comprised of administrator device 208. Administrator device 208 is preferably a standalone workstation such as the Dell Precision 3650 Tower Workstation. Administrator device 208 includes application 209, which coordinates communication between client device 210, system server 212 and the administrator device, as will be further described. Administrator device 208 is operatively connected to network 206.

EEG device 204 is operatively connected to headset 202, which will be further described. EEG device 204 collects and records EEG waveform signals and sends them to the client device.

System server 212 is connected to administrator device 208 through network 206. System server 212 includes application 213, which is resident in local memory. Application 213 conducts signal analysis, generates reports, which are stored in and accessed from database 214, as will be further described.

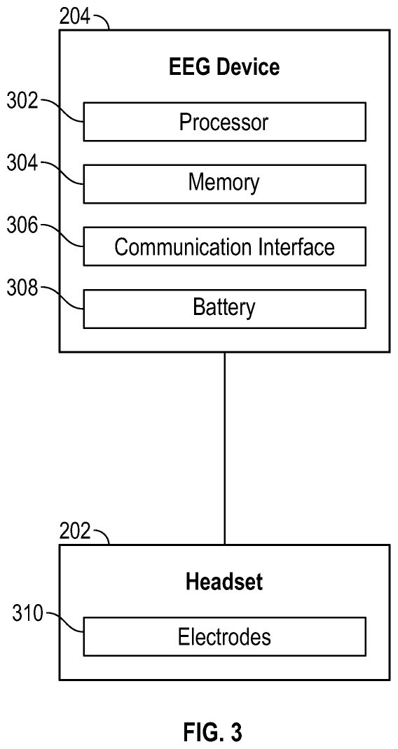

Referring then to FIG. 3, a preferred embodiment of EEG device 204 and headset 202 will be further described. EEG device 204 is comprised of processor 302, memory 304, communication interface 306, and battery 308. EEG device 204 is connected to headset 202 which is comprised of electrodes 310. In a preferred embodiment, the EEG device and the headset are both incorporated in the same unit, namely the SmartingPRO available from mBrainTrain, LLC of Belgrade, Serbia.

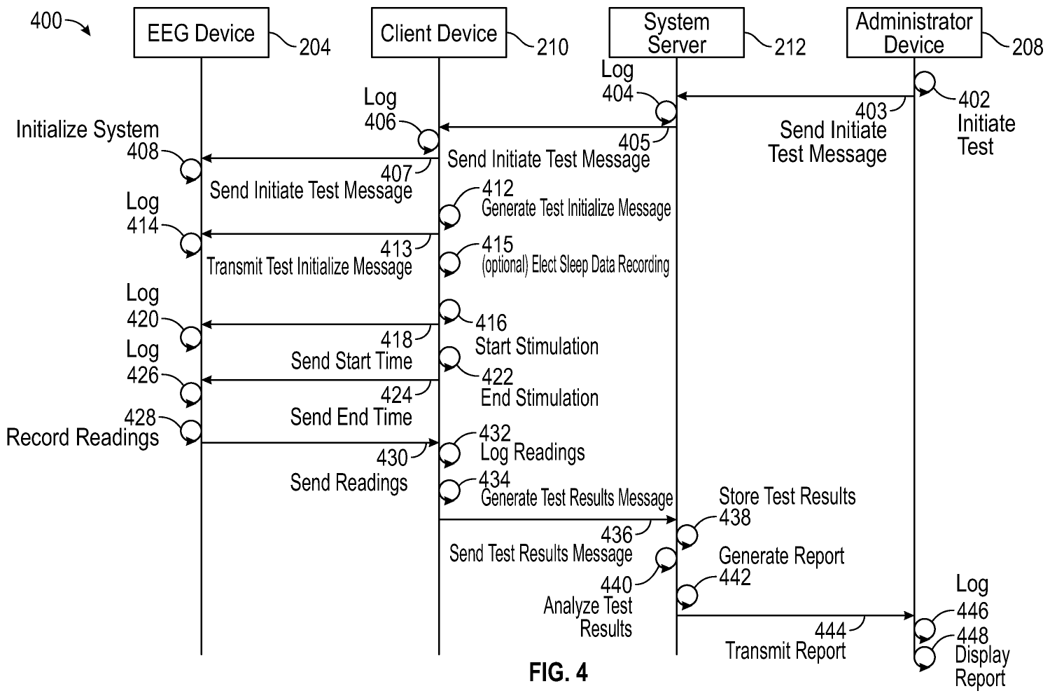

Referring then to FIG. 4, preferred method 400, operable on system 100 and system 200, will be further described.

At step 402, EEG device 204 generates a test initiate message. The test initiate message includes an election of either a stimulus based study or a sleep study.

At step 403, the initiate test message is sent from administrator device 208 to system server 212. At step 404, the system server logs the initiate test message. At step 405, the system server forwards the initiate test message to client device 210. At step 406, client device 210 logs the initiate test message. At step 407, client device 210 sends the initiate test message to EEG device 204. At step 408, the EEG device initializes the system.

At step 412, client device 210 generates a test initialize message. At step 413, client device 210 sends the test initialize message to EEG device 204. At step 414, EEG device 204 logs the test initialize message.

At step 415, optionally, client device 210 elects sleep data recording. In a preferred embodiment, sleep data recording employs scoring for sleep staging NREM 1-3 stages, REM (including onsite, total time spent in each stage, total duration, total time) calculation of sleep efficiency, as well as spindle count, and sleep spindle density. If sleep data recording is elected then stimulation steps 416, 418, 420 and 422 are not performed and the method moves to step 428.

At step 416, if a stimulus based study has been elected, client device 210 starts a stimulation routine in which stimulation is periodically provided to the patient synchronized to start and stop times. The stimulation may be aural, visual or tactile. At step 418, if a stimulus based study has been elected, client device 210 sends the stimulation start time to EEG device 204. At step 420, if a stimulus based study has been elected, EEG device 204 logs the stimulation start time. At step 422, client device 210 ends the stimulation. At step 424, if a stimulus based study has been elected, client device 210 sends the stimulation end time to EEG device 204. At step 426, if a stimulus based study has been elected, EEG device 204 logs the stimulation end time.

At step 428, EEG device 204 records EEG readings from the electrodes attached to the patient. At step 430, EEG device 204 sends the recorded EEG readings to client device 210. At step 432, client device 210 logs the recorded EEG readings.

At step 434, client device 210 generates a test results message. The test results message includes the EEG readings, and a metadata set identifying the EEG system, the client device and the patient. A time signature file, synchronized with the EEG readings is also included in the report. At step 436, client device 210 sends the test results message to system server 212. At step 438, system server 212 stores the test results. At step 440, the stored test results are analyzed, as will be further described. At step 442, the system server generates a report. At step 444, the system server transmits the report to administrator device 208. At step 446, the administrator device stores the report. At step 448, the report is displayed.

The preferred embodiment of this invention, based on scalp-electrode measurements of continuous EEG activity describes the current and most cost-effective method for objectively evaluating potential change in brain activity associated with pre- and post-treatment states for neuropsychiatric disorders, and other training paradigms associated with alterations in behavior. As a secondary preferred embodiment with respect to time efficiency and patient comfort, MEG is also a planned option. The preferred embodiment of the invention disclosed herein records, digitizes, detects, processes, and analyzes spontaneous and/or evoked EEG responses of a patient/subject to sensory (auditory, visual, tactile, olfactory, or other) stimuli. The sensory stimulus is repetitively applied via the modality-appropriate transducer. Stimuli may be presented to one or both ear(s), visual field(s), or appendage(s) for tactile stimulation. However, the preferred embodiment is binaural auditory and full field visual. Tactile stimulation in the preferred embodiment is a single point of stimulation.

For the preferred embodiment, EEG measurements of electrode impedance are taken at brain signal amplifier impedance check and are measured at the start and just prior to the end of each recording. Measurement and control of electrode impedances, or the electrical conductivity between the EEG electrode and this scalp, provides a first step quality control for the measured brain activity. All modern EEG amplifiers are capable of recording brain activity at high impedances. However, low impedance contact significantly reduces the contribution of radiated electrical noise (electrical line noise, and other radiate sources) to the targeted goal of measuring brain activity from the scalp.

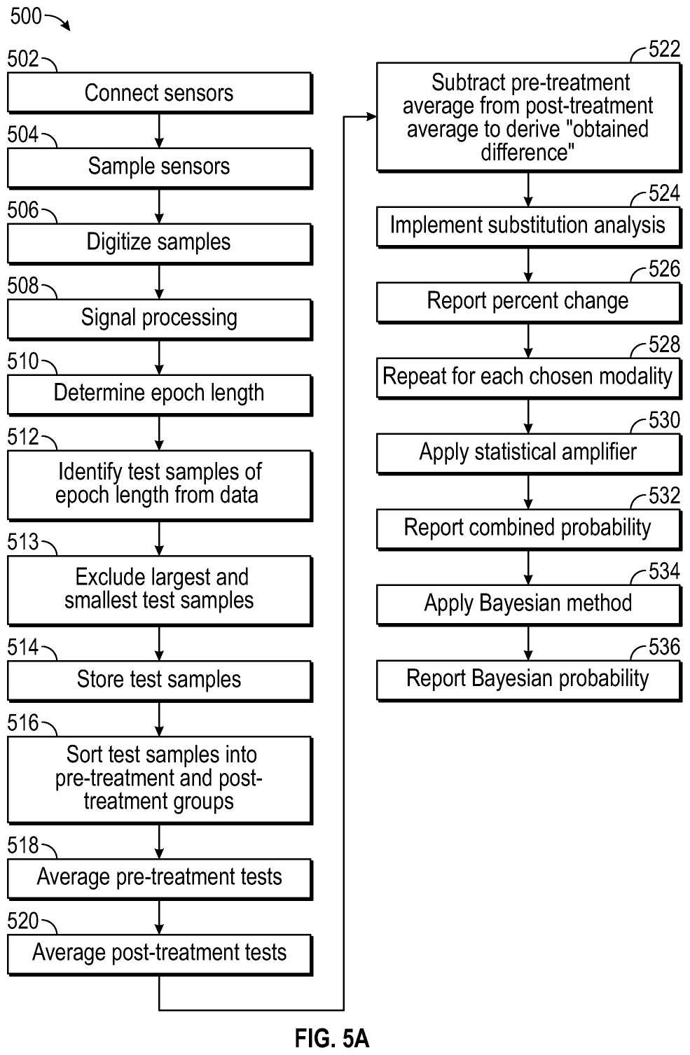

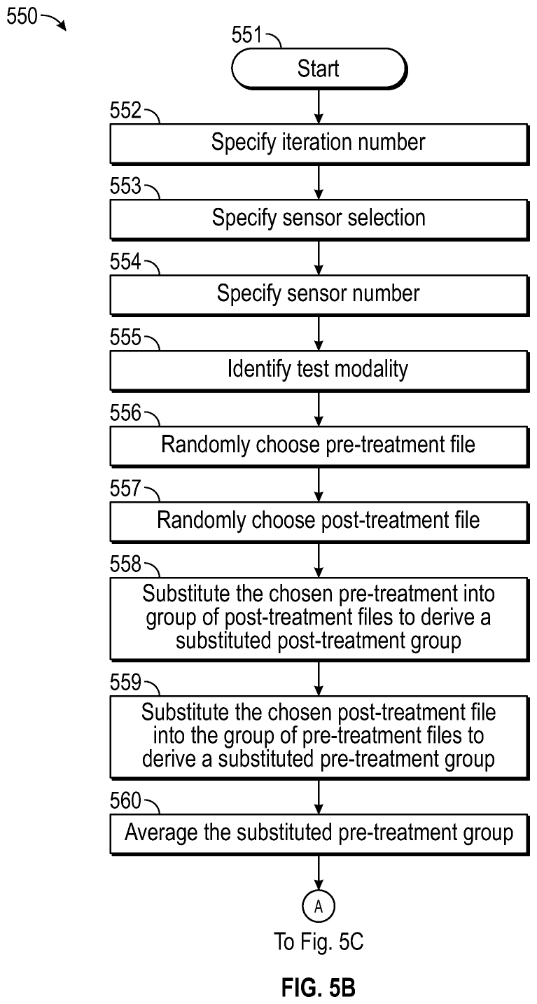

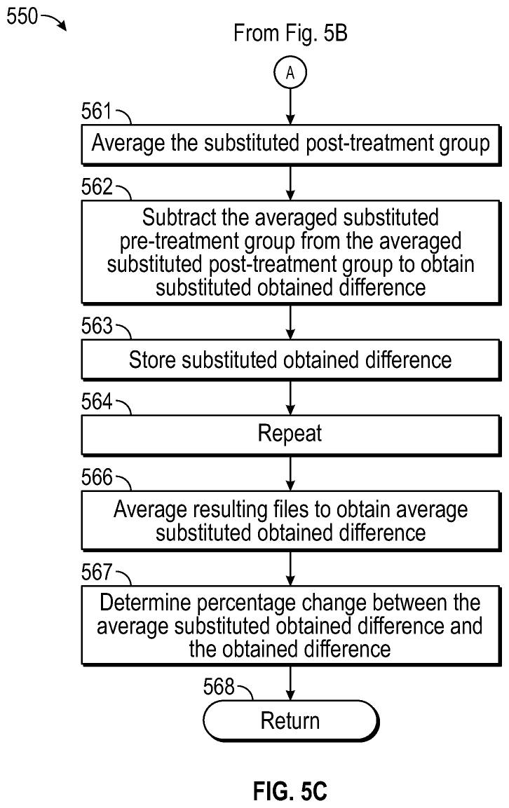

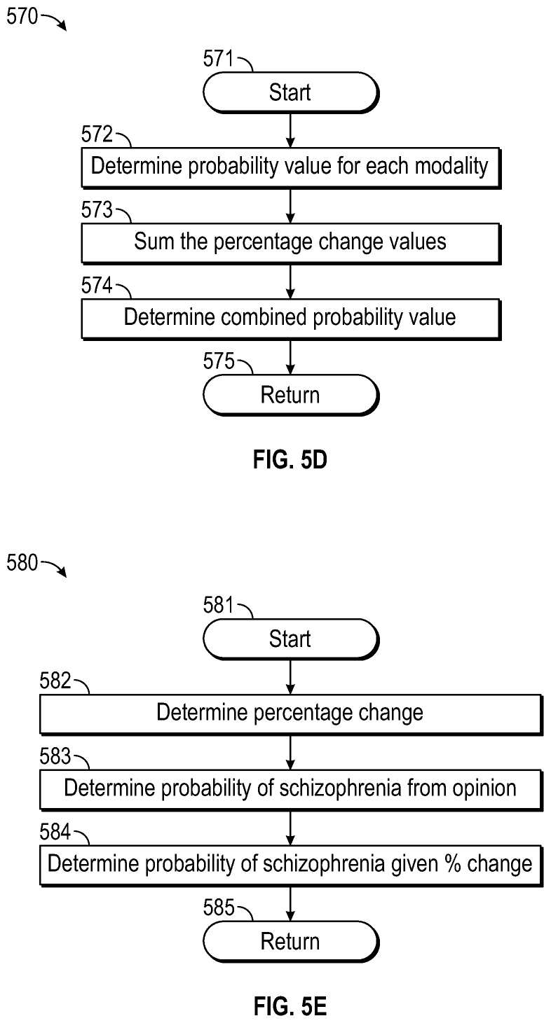

Referring then to FIG. 5A, preferred method 500 for determining percentage change between pre- and post-treatment will be further described.

At step 502, EEG electrode sensors of a brain signal transducer are placed at multiple spatial locations on the scalp to provide adequate spatial sampling so that positive and negative polarities for each sensory modality is represented (opposite ends of the equivalent source of modeled dipole representation of brain activity). Minimally, this is anticipated to be five electrodes, as well as a reference and ground electrode. If only one sensory modality will be tested, at least two electrodes (plus reference and ground) can be placed on the scalp, spatially positioned to be optimally sensitive for measuring both positive and negative polarities of electrical activity from the presumed cortical areas involved in generating the brain response of interest.

At step 504, positive and negative displacements of EEG voltage are continuously sampled and measured to both spontaneous and evoked brain activity.

At step 506, the data points are digitized. The digitized values represent polarity of activation and amplitude of the brain activity for each point in time measurement determined by the digitization rate. The digitization rate of the EEG data indicates how many times per second the analog signal recorded from scalp electrodes is converted to a digitized value for storage. In the preferred embodiment, this sampling rate is at least 500 Hz. However, it may range from 100 Hz to 10,000 kHz or higher. EEG activity is recorded continuously, with a synchronized digital time marker placed at the of each stimulus beginning and ending of each stimulus presentation. The digital time markers facilitate a precise time-locked measurement between the presentation of the stimulus and the onset in the EEG signal.

At step 508, initial stages of signal processing are applied, including but not restricted to baseline offset correction, filtering, and artifact suppression or rejection.

For spontaneous EEG without a defined stimulus to evoked brain activity, the measurement recording is divided into time epochs of a fixed duration. At step 510, epoch length for each test file is determined. The duration of these epochs represents the minimum frequency that a wavelength can be resolved for analysis. The epoch or time window must be a minimum of 500 ms or 0.5 seconds to fully resolve a waveform that contains a 2 Hz or cycles per second frequency. To resolve a 1 Hz or 1 cycle per second waveform, duration of the time epoch of brain activity should be 1 second. The limit for high frequency measurement is constrained by the digitization rate. Based on Nyquist sampling theory, the digitization rate must be at least twice the maximum frequency of interest. That is, to measure a brain signal at 100 Hz requires a minimum digitization rate of 200 Hz, or 200 digitization points per second. Most of the brain signals of interest fall into the frequency range of 0-100 Hz. Thus, epoch durations for spontaneous EEG are consecutive time intervals 1 second epochs of data or longer.

For evoked brain activity, the epoch duration depends on the brain signal of interest and may be as short or as long as desired and may contain just a single stimulus event or multiple stimulus events depending on the proposed method of data analysis. With evoked brain activity, an epoch is defined as having a pre-stimulus interval, defined as brain activity occurring prior to the time-mark delineating stimulus onset (or offset) and a post-stimulus interval. The duration of the post-stimulus interval is defined by the offset of the brain activity waveform components of interest and on the duration (frequency content) of these components.

The epoch duration for spontaneous activity depends on the nature of a passive versus active task (e.g., but not restricted to mentally relaxing or performing math calculations, or any other activity). For stimulus event evoked epochs, the epoch duration depends on the modality of stimulus presentation and, in the specific case of evoked activity, the time (latency) of the evoked brain activity component of interest.

At step 512, for spontaneous and for evoked brain activity, individual test files of epoch length are extracted from the digitized file containing the original continuous EEG/MEG data.

At step 513, approximately 10% of the test samples with extremely high amplitudes and approximately 5% of the test samples with extremely low amplitude are removed from the data files. These high amplitude and low amplitude test samples are not representative of the bulk of the data and may contain residual activities such as eye blinks or excessive body movements, facial muscle contraction (for high amplitudes) that have remained in the time series EEG data after the stages of signal processing. Other percentages may be employed.

At step 514, the individual test files are stored in memory.

At step 516, the individual test files are sorted into groups of pre-treatment and post-treatment groups, and according to the test number (s-number) of the stimulus presentation in each test series.

Individual spontaneous or evoked time epochs of brain activity measured separately or as part of a continuous EEG acquisition using appropriate EEG amplifiers and data acquisition software in responses to one or to a plurality of sensory stimuli (auditory, visual, tactile, olfactory) are collected in pre- and in post-treatment (active or placebo) conditions. Each time a test file is extracted from the continuous recording of EEG brain activity, it contains a single spontaneous or stimulus-evoked sample of brain activity at as few as one electrode/sensor location to a plurality of electrode locations distributed across the scalp. In another embodiment of the invention, individual test files extracted from a measurement (recording) of spontaneous non-evoked EEG/MEG brain activity are collected in pre- and post-treatment (active or placebo) and subjected to re-combination statistical analysis for evidence of change as will be described later.

At step 518, the individual test files in the pre-treatment group are averaged. At step 520, individual test files in the post-treatment group are averaged. In a preferred embodiment, time-series individual test files of continuously acquired EEG consisting of spontaneous and/or evoked and/or induced brain activity are averaged in the time or in the frequency domain based on collection time points determined by pre- vs post-treatment administration (with the understanding that the post-treatment measurement may be obtained from an individual on an active treatment agent or an inert, non-active placebo treatment). Later, comparisons are then based on a point-by-point analysis of each of the averaged test files.

At step 522, the average of the pre-treatment file set is subtracted, point-by-point, from the average post-treatment file set to derive the “obtained difference”.

At step 524, a substitution analysis of the pre- and post-treatment files sets is conducted, as will be further described. Note that these analyses apply not only to amplitude changes in brain activity measures but also to changes in peak latency of brain activity components in the time domain as well as changes in peak frequency components in the time domain.

At step 526, a percentage of change is calculated between the baseline pre-treatment and post-treatment tests and the substitution pre-treatment and post-treatment tests.

At step 528, the percent change is repeated for each chosen modality.

At step 530, a statistical amplifier is applied, as will be further described.

At step 532, the combined probability is reported.

At step 534, a Bayesian method is applied, as will be further described.

At step 536, a Bayesian probability is reported.

Referring then to FIGS. 5B and 5C, preferred method 550 of implementing step 524 will be further described.

At step 551, the method begins. At step 552, an iteration number is specified. In the preferred embodiment, the iteration number is 1,000, but of course may be larger or smaller. Smaller iteration numbers produce quicker results, computationally; however, larger numbers produce more exact results, at the expense of computational efficiency.

At step 553, a sensor selection is made. In the preferred embodiment, an EEG headset having thirty-two (32) sensors is employed. Of these 32 sensors, a subset of at least 10 is chosen. Subsets as small as 1 or as large as 32 may also be chosen.

At step 554, optionally, a particular sensor number from the sensor selection subset is chosen.

At step 555, a test modality is selected. In situations where multiple test modalities are used for a single patient or a patient group, this step allows the specification of a particular modality from the group of modalities.

For EEG/MEG measures of brain activity, time-series averages are typically based on tens, to hundreds, to thousands of individual epoch files. The total number of possible combined averages increases exponentially as the numbers of epoch files increase. An option is to specify a random subset of all total combinations to be calculated, which can be executed very rapidly, on the order of seconds to minutes. Such analysis is performed for all digitized time point measurements of the brain activity from each sensor channel. Results provide a time (or frequency) point-by-point probability estimate of significant change across all sensors. Optionally, a subset of sensors or a region of interest of sensors (e.g., for EEG, midline electrodes) may be selected for analysis.

At step 556, a pre-treatment epoch file is randomly chosen and extracted from the pre-treatment file group.

At step 557, a post-treatment epoch file is randomly selected and extracted from the post-treatment file group.

At step 558, the selected pre-treatment epoch file is substituted into the group of post-treatment epoch files in place of the randomly selected post-treatment epoch file to derive a substituted post-treatment group.

At step 559, the randomly selected post-treatment file is substituted into the pre-treatment file group in place of the randomly selected pre-treatment file, to derive a substituted pre-treatment group.

At step 560, the substituted pre-treatment group is averaged. In a preferred embodiment, the averaging operation is point by point across each epoch file.

At step 561, the substituted post-treatment group is averaged. In a preferred embodiment, the averaging operation is point by point across each epoch file.

At step 562, the average substituted pre-treatment group is subtracted, point-for-point from the averaged substituted post-treatment group to obtain a substituted obtained difference.

At step 563, the substituted obtained difference is stored in a substituted obtained difference group in the database.

At step 564, steps 556 through 563 are repeated for the predetermined number of iterations, storing a new substituted obtained difference file into the substituted obtained difference group.

At step 566, all test files in the substituted obtained difference group are averaged, point-for-point, to obtain the average substituted obtained difference.

At step 567, the percentage change is determined between the average substituted obtained difference and the obtained difference.

At step 568, the subroutine returns the percent change.

Referring to FIG. 5D, preferred additive combination method 570 of step 530 will be further described.

At step 571, the method begins.

At step 572, the probability value for each modality is determined.

At step 573, the sum of the percent change values for each modality are summed according to the following equation.

$${S}_{n} = {\sum }_{1}^{n} modality \% change$$

Where:
n=number of modalities; and,
S=sum of probability.

At step 574, the combined probability value is determined according to the following equation.

$${P}_{c} = \frac{{s}_{n}}{n !}$$

Where:
P_{c}=combined probability;
Sₙ=sum of probability value; and,
n=number of modalities.

At step 575, the method returns the combined probability value.

The preferred embodiment of the invention includes multiple levels of analysis. In addition to traditional parametric statistical analysis, the preferred embodiment also includes for the purposes of pre- versus post-treatment analysis non-parametric as well as Bayesian statistical approaches that are applied for both individual and group level comparisons, as will be further described.

Referring to FIG. 5E, preferred Bayesian method 580, of step 534 will be further described.

At step 581, the method begins.

At step 582, the percent change is determined, as previously described.

At step 583, the probability of schizophrenia is determined from a clinician opinion.

At step 584, the following equation is employed to determine the probability of schizophrenia given the percent change.

$$P ( A \vert B ) = \frac{P ( B \vert A ) P ( A )}{P ( B )}$$

Where:
P(A|B) is the probability of schizophrenia given the percent change;
P(B|A) is the probability of a percent change given the opinion of schizophrenia;
P(A) is the probability of schizophrenia without respect to given conditions; and,
P(B) is the probability of the percent change.

At step 585, the method returns the probability of schizophrenia given the percent change.

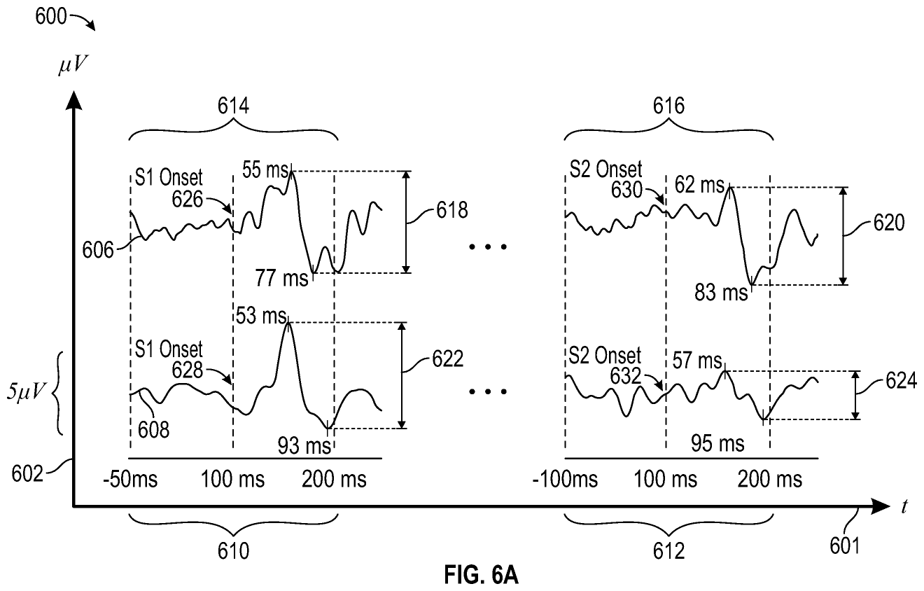

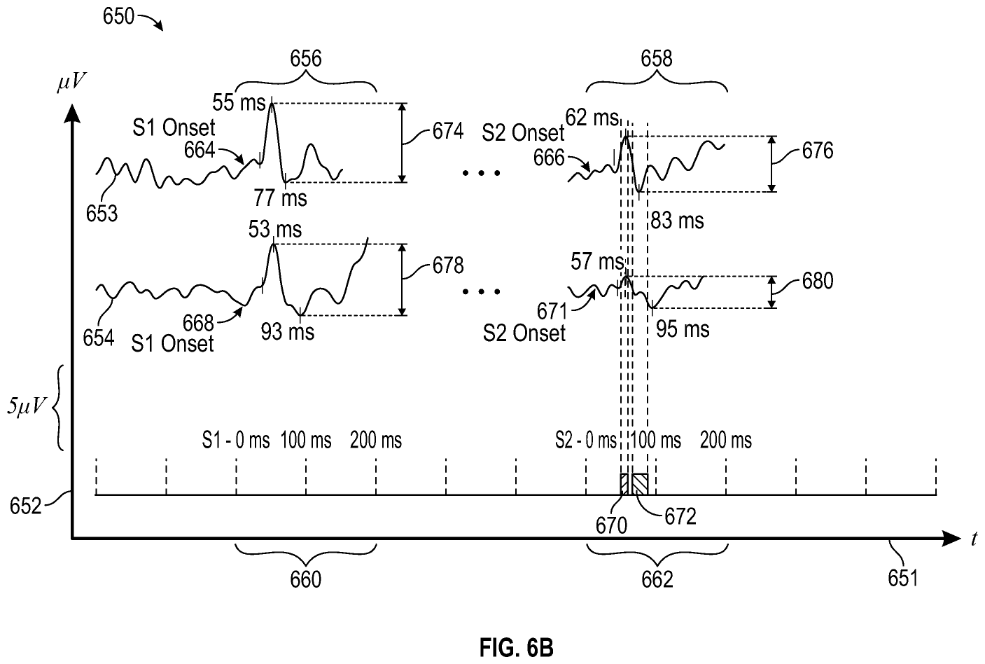

Referring to FIG. 6A, test results from application of the described substitution analysis will be further described. In non-psychiatric normal populations, the magnitude of the P50 component is significantly reduced for P50(S2) relative to P50(S1). This inhibition is often 30-50% or larger. This suppression has been interpreted and described as an attentional gating process, in which the brain activity generating the P50(S1) inhibits or suppresses the brain activity generating P50(S2).

Graph 600 is presented with x-axis 601 indicating time and y-axis 602 indicating microvolts. Active stimulus was employed in each case as a 100 microsecond DC offset auditory evoked potential.

Curve 606 shows an EEG file applying the described substitution analysis for epoch 614 and epoch 616, taken on day 4 of active treatment. Epoch 614 includes S1 onset at 626. Epoch 616 includes S2 onset at 630. Curve 608 shows an EEG file applying the described substitution analysis for epoch 610 and epoch 612 taken on day 14 of active treatment. Epoch 610 includes S1 onset at 628. Epoch 612 includes S2 onset at 632.

Curve 606 for epoch 614 indicates a P50(S1) suppression 618. Likewise, curve 606 for epoch 616 shows a P50(S2) suppression 620. The percent change between suppression 620 and suppression 618 is indicated by the described substitution analysis to be about 5%.

Curve 608 at epoch 610 shows a P50(S1) suppression 622 at day 14 of active treatment. Curve 608 also shows a P50(S2) suppression of 624. The percent change between suppression 624 and suppression 622 by the described substitution analysis is indicated to be about 46%.

FIG. 6A indicates a substantial normalization of inhibition from day 4 of active treatment to day 14 of active treatment. The implementation of substitution analysis enables easy recognition of the change by eliminating substantial noise interference in the signals.

Referring then to FIG. 6B, test results from application of the described substitution analysis will be further described. Active stimulus was employed in each case as a 100 microsecond DC offset auditory evoked potential.

Graph 650 is presented with x-axis 651 indicating time and y-axis 652 indicating microvolts.

Curve 653 shows an EEG file applying the described substitution analysis for epoch 656 and epoch 658 taken on day 0 of active treatment. Epoch 656 includes S1 onset at 664. Epoch 658 includes S2 onset at 666. Curve 654 shows an EEG file applying the described substitution analysis for epoch 660 and epoch 662, taken on day 14 of active treatment. Epoch 660 includes S1 onset at 668. Epoch 662 includes S2 onset at 671.

Curve 653 for epoch 656 indicates a P50(S1) suppression 674. Likewise, curve 653 for epoch 658 shows a P50(S2) suppression 676. The percent change between suppression 676 and suppression 674 is indicated by the described substitution analysis to be about 30%.

Curve 654 at epoch 660 indicates a P50(S1) suppression 678 at day 14 of active treatment. Curve 654 also shows a P50(S2) suppression of 680. The percent change between suppression 680 to suppression 678 is indicated by the described substitution analysis to be about 52%.

FIG. 6B indicates a substantial normalization of inhibition from day 0 of active treatment to day 14 of active treatment. The implementation of the substitution analysis, again enable easy recognition of the change by eliminating substantial noise interference in the signals.

Boxes 670 and 672 indicate a comparison of the peak-to-trough amplitude of P50(S2) suppression between day 0 and day 14. Box 670 shows a statistically significant S2-P50 contrast for 51-69 ms when comparing curve 653 with curve 654. Box 672 shows a statistically significant S2-P50 contrast for 71-98 ms when comparing curve 653 with curve 654. The substitution analysis shows that amplitude at the peak of P50 from 51-69 ms and the trough from 71-98 ms are significantly larger, p>0.01 level at day 0 than at day 14. This difference is consistent with a significant increase in P50 suppression following two weeks of active drug administration for a product known to improve psychotic symptoms in schizophrenia. A similar result was obtained for substitution analysis run in the frequency domain, where significant differences (p<0.01) between 20-26 Hz.

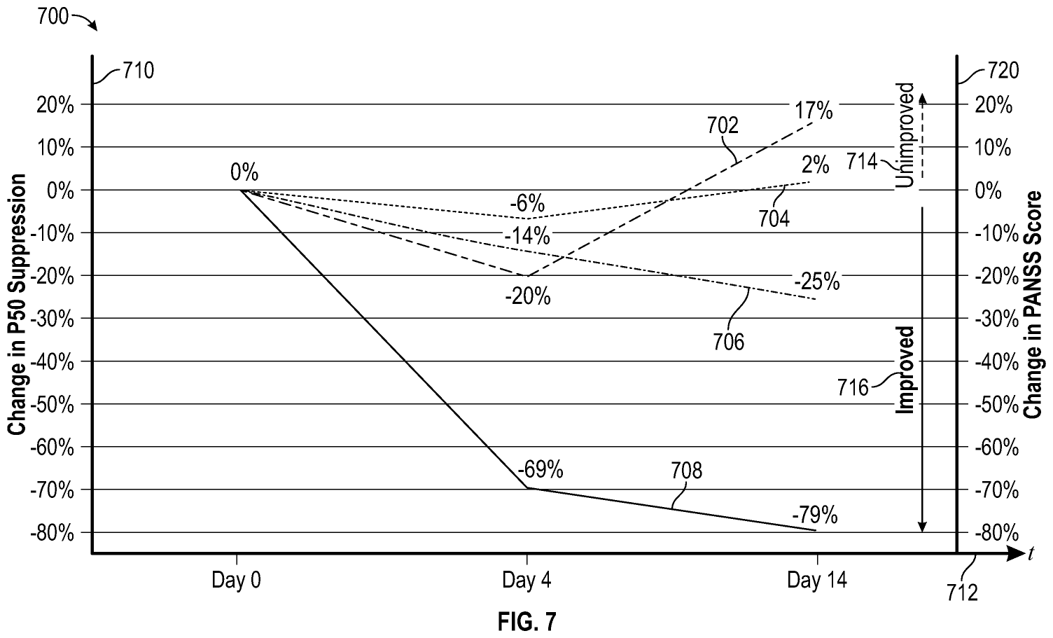

Referring then to FIG. 7, a summary of results changes in P50 attenuation and PANSS scores for day 4 and day 14 will be further described.

Graph 700 contains data from a double-blind IRB-cleared study showing results obtained from all patients tested. Graph 700 illustrates changes in P50 suppression by percentage on y-axis 710 and PANSS score by percentage on y-axis 720, both as a function of time on x-axis 712, from day 0, day 4 and day 14. P50 suppression is calculated by measuring the reduction in amplitude in microvolts of the response from the first stimulus to the second stimulus.

Arrow 714 illustrates that an increase in PANSS scores or unimproved symptoms. Arrow 716 illustrates that a decrease PANSS scores or improved symptoms.

The patients assigned to the active drug (four patients) are represented by active drug PANSS score 706 and active drug P50 suppression 708. Patients assigned to the placebo (two patients) are represented by placebo P50 suppression 702 and placebo PANSS score 704. For the patients on active drug, average improvements from day 0 to day 14 were 25% for active drug PANSS score 706 and 79% for active drug P50 suppression 708. For the two patients on placebo, there was far less change by day 14, showing a small worsening for both placebo PANSS score 704 of about 2% and placebo P50 suppression 702 of about 17%.

Statistically, the changes in PANSS scores were significant using both parametric statistics (p<0.045) and substitution analysis approach (p=0.003). Due to the high level of difference in P50 suppression across patients, a parametric t-test did not show significance (p<0.13). However, due to the complete separation of active drug and placebo results, the substitution analysis for difference in means between the groups did show significant suppression (p=0.025).

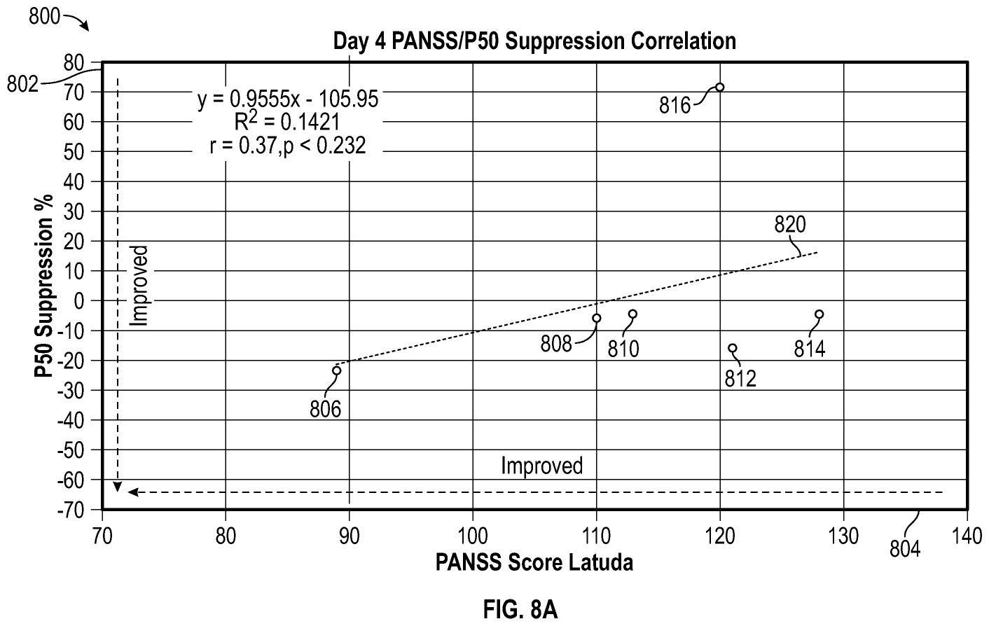

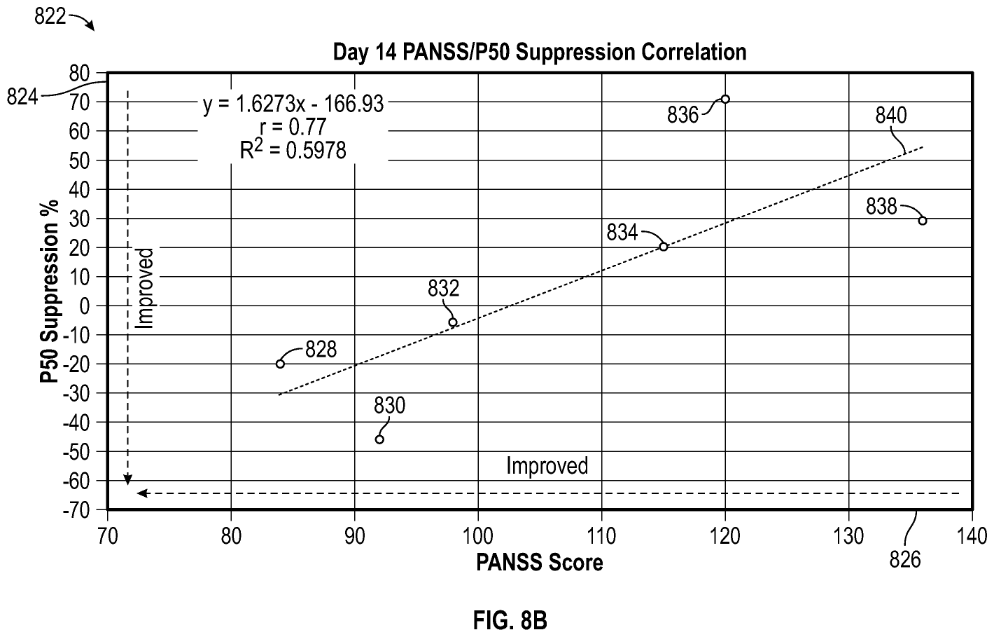

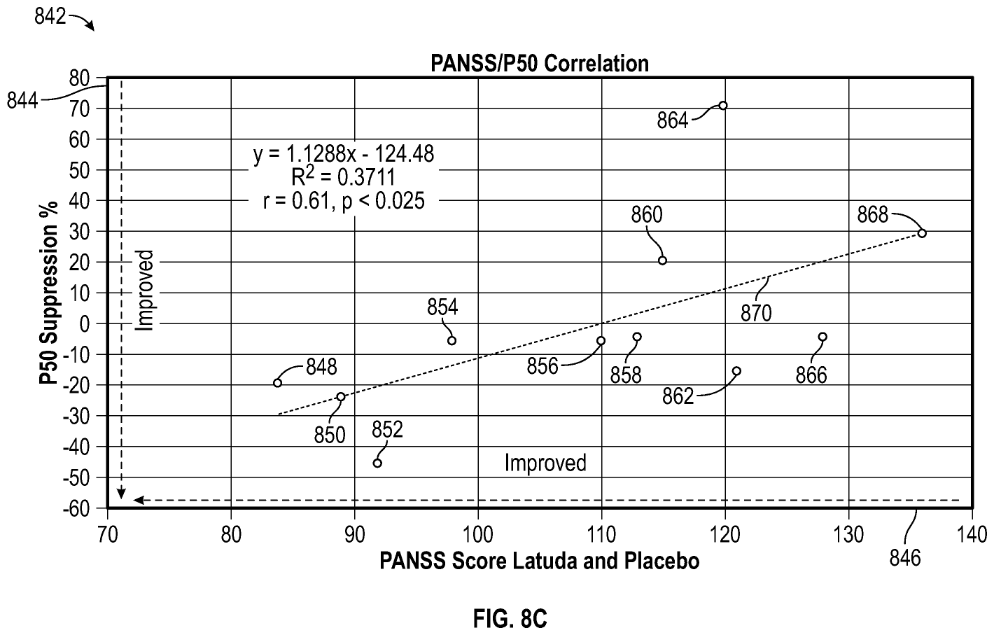

Referring then to FIGS. 8A, 8B, and 8C graphs from actual tests performed showing a correlation between P50 suppression and PANSS scores will be further described.

FIGS. 8A, 8B, and 8C indicate test results for all individual patients, showing a correlation between P50 suppression, measured as a percentage change from baseline, and PANSS scores, first for patients in the active drug group. For the active drug group, using a simple Pearson's statistic, the correlation of 0.37 (p>0.05) at day 4 increased to 0.77 (p<0.04) day 14 post-randomization. When the placebo data was added the correlation on Day 14 was 0.61 (p<0.035).

Referring then to FIG. 8A, graph 800 represents a correlation between P50 suppression and PANSS scores for all individual patients receiving active treatment at day 4. PANSS scores for active treatment is reflected across x-axis 804 and P50 suppression is reflected across y-axis 802. Data points 806, 808, 810, 812, 814, and 816 each represent an individual patient in the active drug group. As PANSS scores increase across x-axis 804, P50 suppression increases across y-axis 802. Best fit line 820 generally represents an improvement trend with PANSS scores and P50 suppression.

Referring then to FIG. 8B, graph 822 represents a correlation between P50 suppression and PANSS scores for all individual patients receiving active treatment at day 14. PANSS scores for active treatment is reflected across x-axis 826 and P50 suppression is reflected across y-axis 824. Data points 828, 830, 832, 834, 836, and 838 each represent an individual patient in the active drug group. As PANSS scores increase across x-axis 826 P50 suppression increases across y-axis 824 and shows an improvement over day 14 active treatment. Best fit line 840 generally represents an improvement trend with PANSS scores and P50 suppression.

Referring then to FIG. 8C, graph 842 represents a correlation between P50 suppression and PANSS scores for all individual patients receiving active and placebo treatment at day 14. PANSS scores for active treatment and placebo is reflected across x-axis 846 and P50 suppression is reflected across y-axis 844. Data points 848, 850, 852, 854, 856, 858, 860, 862, 864, 866, and 868 each represent an individual patient in either the active drug group or the placebo group. As PANSS scores increase across x-axis 846 P50 suppression increases across y-axis 844 and shows improvement but the improvement was slightly less than the improvement at day 14 active treatment. Best fit line 870 generally represents an improvement trend with PANSS scores and P50 suppression.

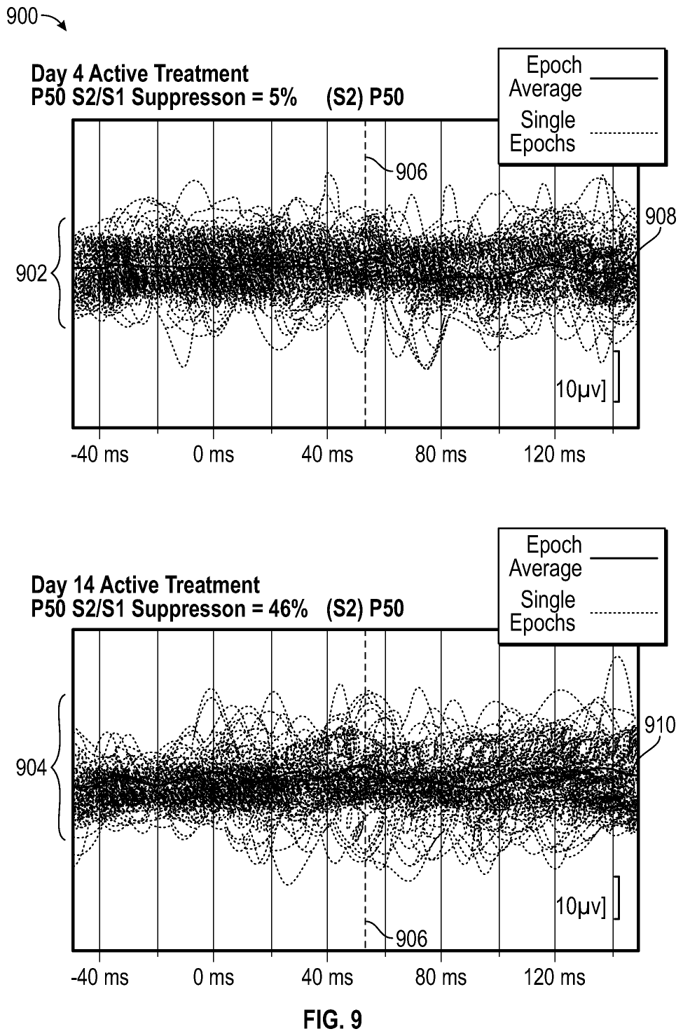

Referring then to FIG. 9, an averaged evoked EEG auditory P50 brain activity and 80+ individual time-series epochs at day 4 and day 14 of drug treatment will be further described.

Graph 900 shows averaged time series epoch 908 at day 4 and averaged time series epoch 910 at day 14 superimposed on individual 80+ time series epoch 902 at day 4 and individual 80+ time series epoch 904 at day 14 for a patient in the active drug group. P50(S2) peak is indicated at demarcation line 906. The substitution analysis based on a randomly selected subset of all possible combinations of averaged pre-treatment and post-treatment files shows significant decreases in P50 related brain activity from day 4 to day 14 post drug administration for an individual subject.

The present invention can be executed by other than the stated preferred embodiments. The preferred embodiments are included for illustration purposes only and do not define a singular set of parameters. Other similar parameters or stimulus presentation paradigms can be included as part of this invention that will provide similar outcomes or augment existing outcomes as described above. It should be understood similar parameter values can be used without substantively deviating from the intent or scope of the invention.

1. A system for implementing a substitution analysis comprising:
a set of patient electrodes;
an EEG device, including a first memory, operatively connected to the set of patient electrodes;
a signal processing computer, including a second memory, operatively connected to the EEG device;
a set of instructions, resident in the first memory and the second memory, that when executed cause the system to:
identify a set of signal test samples;
divide the set of signal test samples into a pre-treatment group and a post-treatment group;
point-for-point average the pre-treatment group to derive a pre-treatment average;
point-for-point average the post-treatment group to derive a post-treatment average;
subtract the pre-treatment average from the post-treatment average to derive an obtained difference;
implement a substitution analysis on the pre-treatment group and the post-treatment group to derive an average substituted obtained difference;
determine a percent change between the average substituted obtained difference and the obtained difference; and,
report the percent change.

2. The system of claim 1 wherein the set of instructions further comprises instructions that when executed cause the system to:
identify a largest 10% of the set of signal test samples;
identify a smallest 5% of the set of signal test samples;
exclude the largest 10% of the set of signal test samples; and,
exclude the smallest 5% of the set of signal test samples.

3. The system of claim 1 wherein the set of signal test samples is drawn from one of the group of EEG signal test samples, MEG signal test samples and sleep data signal test samples.

4. The system of claim 1 wherein the set of signal test samples is drawn from one of the group of a set of group data and a set of individual data.

5. The system of claim 1 wherein the set of instructions further comprises instructions that when executed cause the system to apply a statistical amplifier.

6. The system of claim 5 wherein the step of applying a statistical amplifier further comprises:
determining a set of probability values for a set of modalities;
summing the set of probability values; and,
determining a combined probability value.

7. The system of claim 1 wherein the set of instructions further comprises instructions that when executed cause the system to apply a Bayesian probability.

8. The system of claim 7 wherein the step of applying a Bayesian probability further comprises:
determining a first probability of schizophrenia from a clinical opinion; and,
determining a second probability of schizophrenia based on the percent change and the first probability.

9. The system of claim 1 further comprising a client processor, including a third memory, operably connected to the EEG device and wherein the set of instructions is further resident in the first memory, the second memory and the third memory and further comprising instructions that when executed cause the system to:
initiate a stimulation routine; and,
terminate the stimulation routine.

10. The system of claim 9 wherein the client processor is wirelessly connected to the EEG device.

11. The system of claim 9 wherein the client processor is wirelessly connected to the signal processing computer through a wide area network.

12. The system of claim 9 wherein the stimulation routine further comprises one of a group of visual stimulation, audio stimulation and tactile stimulation.

13. The system of claim 1 wherein the set of patient electrodes further includes 1 to 32 patient electrodes.

14. A method of implementing a substitution analysis comprising:
providing a set of patient electrodes;
providing an EEG device, including a first memory, operatively connected to the set of patient electrodes;
providing a signal processing computer, including a second memory, operatively connected to the EEG device;
providing a set of instructions, resident in the first memory and the second memory, that execute the steps of:
identifying a set of signal test samples;
dividing the set of signal test samples into a pre-treatment group and a post-treatment group;
point-for-point averaging the pre-treatment group to derive a pre-treatment average;
point-for-point averaging the post-treatment group to derive a post-treatment average;
subtracting the pre-treatment average from the post-treatment average to derive an obtained difference;
implementing a substitution analysis on the pre-treatment group and the post-treatment group to derive an average substituted obtained difference;
determining a percent change between the average substituted obtained difference and the obtained difference; and,
reporting the percent change.

15. The method of claim 14 further comprising providing instructions, resident in the first memory and the second memory, that execute the steps of:
identifying a largest 10% of the set of signal test samples;
identifying a smallest 5% of the set of signal test samples;
excluding the largest 10% of the set of signal test samples; and,
excluding the smallest 5% of the set of signal test samples.

16. The method of claim 14 further comprising drawing the set of signal test samples from one of the group of EEG signal test samples, MEG signal test samples and sleep data signal test samples.

17. The method of claim 14 further comprising drawing the set of signal test samples from one of the group of a set of group data and a set of individual data.

18. The method of claim 14 further comprising providing instructions, resident in the first memory and the second memory, that execute the step of applying a statistical amplifier.

19. The method of claim 18 wherein the step of applying a statistical amplifier further comprises:
determining a set of probability values for a set of modalities;
summing the set of probability values; and,
determining a combined probability value.

20. The method of claim 14 further comprising providing instructions, resident in the first memory and the second memory, that executes the step of applying a Bayesian probability.

21. The method of claim 20 wherein the step of applying a Bayesian probability further comprises:
determining a first probability of schizophrenia from a clinical opinion; and,
determining a second probability of schizophrenia given the percent change.

22. The method of claim 14 further comprising providing a client processor, including a third memory, operably connected to the EEG device, and, providing instructions, resident in the first memory, the second memory and the third memory that execute the steps of:
initiating a stimulation routine; and,
terminating the stimulation routine.

23. The method of claim 22 further comprising wirelessly connecting the client processor to the EEG device.

24. The method of claim 22 further comprising wirelessly connecting the client processor to the signal processing computer through a wide area network.

25. The method of claim 22 further comprising drawing the stimulation routine from one of a group of visual stimulation, audio stimulation and tactile stimulation.

26. The method of claim 14 further comprising drawing the set of patient electrodes from a number of 1 to 32 patient electrodes.
